(19) United States
(12) Patent Application Publication
(10) Pub. No.: US 2003/0144881 A1
(43) Pub. Date: Jul. 31, 2003

(54) Method and program for identifying multiple diluent solutions for use in drug delivery with a healthcare system

(76) Inventors: Kaivan Talachian, Richmond Hill (CA); Sayeh Radpay, Toronto (CA); Meetali Acharya, Burlington (CA); Eric S. Paul, North York (CA)

Correspondence Address: Wallenstein & Wagner, Ltd., 53rd Floor, 311 S. Wacker Drive, Chicago, IL, 60606-6622, US

(21) Appl. No.: 10/237,154

(22) Filed: Sep. 6, 2002

Related U.S. Application Data
(63) Continuation-in-part of application No. 10/135,180, filed on Apr. 30, 2002.
Application No. 10/135,180 is a continuation-in-part of application No. 10/059,929, filed on Jan. 29, 2002.
(60) Provisional application No. 60/377,027, filed on Apr. 30, 2002.
(60) Provisional application No. 60/376,625, filed on Apr. 30, 2002.
(60) Provisional application No. 60/376,655, filed on Apr. 30, 2002.

(51) Int. Cl. A61K 9/22

(52) U.S. Cl. 705/2; 604/892.1

(57) ABSTRACT

A diluent identifying system comprising a computer or computer network for maintaining a database of additives for infusion processes. The system identifying one or more diluents that can be used with each additive.

### RELATED APPLICATIONS

The present application claims the benefit of the following U.S. Provisional Applications: “Medical Delivery and Information System for Use in Health Care Facilities,” Serial No. 60/377,027, filed Apr. 30, 2002; “System and Method for Integrating Structural Clinical Documentation With Point of Care Treatment of a Patient,” Serial No. 60/376,625, filed Apr. 30, 2002; “Infusion Therapy System and Method,” Serial No. 60/376,655, filed Apr. 30, 2002; “System And Method For Supporting Clinical Decisions During Patient Care And Treatment,” Serial No. 60/384,717, filed May 31, 2002; “Nursing Order Workflow System and Method,” Serial No. 60/385,176 filed May 31, 2002; “System And Method For Facilitating Orders During Patient Care And Treatment,” Serial No. 60/384,607, filed May 31, 2002; and incorporates such applications herein by reference.

This application is a continuation-in-part of the following co-pending applications: “Medical Treatment Verification System and Method,” Ser. No. 10/135,180, filed Apr. 30, 2002; “Automated Messaging Center System And Method For Use With a Healthcare System,” Ser. No. 10/159,845, filed May 31, 2002; “Infusion Therapy Bar Coding System and Method,” Ser. No. 10/160,429, filed May 31, 2002; “System and Method for Providing Multiple Units of Measurement,” Ser. No. 10/161,221, filed May 31, 2002; “Healthcare Database Management Offline Backup and Synchronization System and Method,” Ser. No. 10/160,444, filed May 31, 2002; “System And Method For Obtaining Information From A Bar Code For Use With A Healthcare System,” Ser. No. 10/160,565, filed May 31, 2002; “System And Method For Facilitating Patient Care And Treatment,” Ser. No. 10/160,563, filed May 31, 2002; and incorporates such applications herein by reference.

Additionally, the present application is being filed concurrently with and incorporates by reference the following applications: “Method and Program for Providing a Maximum Concentration of a Drug Additive in a Solution,” Ser. No. ______; “Method and Program for Creating Healthcare Facility Order Types,” Ser. No. ______; and “System and Method for Facilitating Time-Based Infusion Orders,” Ser. No. ______.

### TECHNICAL FIELD

The present invention is generally directed to a method and program for identifying a diluent for use with an additive, and more particularly to a computerized method and program designed for implementation with a healthcare facility system for identifying one or more diluents that can be used with a particular additive for an infusion process.

### BACKGROUND OF THE INVENTION

A large variety of drugs can be used for infusion processes for intravenous treatment of a patient. Such drugs, or additives, are added to diluents for the infusion. Typically, more than one diluent can be utilized for any particular additive.

Computerized systems can be used in the healthcare field to provide information management for a healthcare facility. Such systems can be used to provide information relating to drugs or medications used for the treatment of patients. However, pharmacy or other healthcare facility systems on the market today do not allow multiple solutions to be pre-defined as diluents for a given additive during system set up. At best, such systems will simply list all diluents available at the healthcare facility without checking for the compatibility of the diluents with a particular selected additive. Certain combinations of diluents with particular additives may not be safe for the patient or adhere to the healthcare facility's best practices.

### SUMMARY OF THE INVENTION

The present invention is directed to a computerized system for identifying one or more diluents for use with an additive for the treatment of a patient. Preferably, the present computerized system is part of or in communication with, a pharmacy system or some other type of healthcare facility system. The diluent identifying system includes a drug file (which may appear or be displayed as a window on the computer monitor) that provides information concerning a drug or medication which can be prescribed to a patient at the healthcare facility. For infusions or other medical treatments requiring diluting a drug, the drug file has the ability to define one default diluent per drug or additive in the drug file. This ability can be improved to allow for multiple default diluents to be specified for each drug in the drug file, with one diluent being designated as the preferred diluent.

The drug file can be implemented with other features of the pharmacy or healthcare facility system. For example, the system may keep track of drug or medication inventory, prescription ordering and status, patient treatment information etc. The system is preferably used by pharmacists, doctors, nurses and other healthcare providers or clinicians.

In one embodiment of the invention, a diluent identifying system for use with a pharmacy system or healthcare facility system comprises a computer configured to generate information relating to a first additive for use in a patient's therapy. The computer is further configured to provide an identification of a first diluent which may be used with the additive in the patient's therapy. A user of the system can select a particular additive from a plurality of additives stored in a database in the system for use in an infusion process for a patient. The system will provide information relating to the selected additive, and identify a diluent that may be utilized with the selected additive.

In addition to identifying a first diluent, the computer can also be configured to generate an identification of a second diluent different than the first diluent which may be used with the first additive in the patient's therapy. In fact this can be expanded to allow for identification of a plurality of diluents that can be used with a particular additive.

The computer can also be configured to receive information associating a diluent which may be used with the additive. That is, diluents can be added to a the drug file for a particular additive. In this manner the healthcare facility can designate the diluents it deems are the best for use by that facility.

The computer may either stand alone, or instead, include an interface for communication over a computer network. In this manner, the drug file may be accessible to a variety of clinicians anywhere in the healthcare facility.

Additional information concerning use of a particular diluent may be provided. For example, the computer can be configured to generate a default volume for an identified diluent, or a specific maximum concentration.

Additionally, the computer can be configured to generate a note associated with any of the diluents associated with a particular additive. The note may contain information relating to the diluent, such as its properties or information as to how to use of implement the diluent.

The computer may be configured to receive orders for a prescription for the additive for use in the patient's therapy. This would include the ability for the computer to receive information from a user selecting one of the diluents associated with the additive for completing the order for the prescription.

The computer in diluent identifying system can be configured to identify one of diluents associated with a particular additive as a default diluent. Moreover, the system can be modified to allow for multiple diluents to be identified as defaults.

In another embodiment of the invention, a method for identifying a diluent for use with an additive in a pharmacy or healthcare facility system comprises the steps of providing a computer for accessing a database containing data associated with a plurality of additives for use in a patient's therapy, receiving information by the computer selecting a first additive from the plurality of additives; and, displaying a first diluent that can be used with the selected first additive. The method may also include displaying a second diluent, or a plurality of diluents, that can be used with the selected first additive.

The method may also include displaying additional information relating to a diluent, such as a default volume or a maximum concentration. Also, the method may include displaying or providing a link to a note associated with a diluent providing information relating to the diluent.

The method may further include identifying one of the diluents associated with a particular additive as the default diluent. This may be expanded to allow for identifying multiple default diluents.

The method may also include allowing a user of the system the ability of inputting into the database a diluent to be associated with a particular additive into the database. When this additive is selected, the new diluent will also be displayed with any other diluents already associated with the additive.

The method may be used in a standalone computer, or as part of a network of devices.

In yet a further embodiment of the invention, a computer program product for identifying a diluent for use with an additive comprises a computer usable medium having computer readable program code embodied therein, the program code including a first portion for maintaining data relating to a plurality of additives for use in a patient's therapy; and, a second portion for identifying a first diluent for use with each of the plurality of additives. Additionally, the computer program product may comprising a third portion for identifying a second diluent for one or more of the plurality of additives.

The program may also include a portion for indicating a default, and/or a specific maximum concentration volume for a diluent. Similarly, the program may include a portion for generating a note associated with a diluent, the note providing data relating to the diluent.

The program may also include a fourth portion for setting one or more of the diluents as a default solution for use with at least one of the additives. Additional portions of the program can be provided for any of the other functionality described herein.

Further aspects of the invention are disclosed in the detailed description of the preferred embodiment, the drawings and the claims.

### BRIEF DESCRIPTION OF THE DRAWINGS

FIG. 1 is a screen shot of a Drug (Edit) window in accordance with the present invention; and,

FIG. 2 is a portion of the screen shot of FIG. 1.

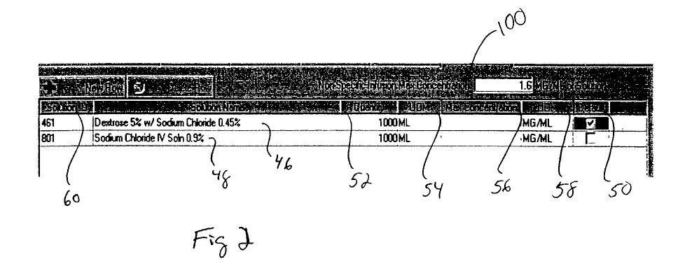

### DETAILED DESCRIPTION OF THE PREFERRED EMBODIMENT

While this invention is susceptible of embodiments in many different forms, there is shown in the drawings and will herein be described in detail preferred embodiments of the invention with the understanding that the present disclosure is to be considered as an exemplification of the principles of the invention and is not intended to limit the broad aspect of the invention to the embodiments illustrated.

The present diluent identification system is preferably used in connection with a medical delivery and information management system for use in hospitals, pharmacies and other similar healthcare facilities (i.e., a healthcare facility system). The preferred healthcare facility system uses a combination of bar coding and wireless technology to support the clinical needs of physicians, pharmacists, nurses and other healthcare personnel (i.e., clinicians). Moreover, the preferred healthcare system allows healthcare professionals and administrators to make better decisions and reduce costs, while improving patient safety and quality of care.

Specifically, among other things, the preferred healthcare system comprises a computer software program integrated with various other healthcare facility systems, that provides for: electronic on-line access to patient information (e.g., medication history, current medications, allergies, reactions) and medication administration information, preferably in the form of an electronic medical record for the patient; instant medication ordering at the patient bedside; clinical screening of medications; an electronic messaging link between physicians, pharmacists and nurses; standardized dosage and special administration instructions; and, an improved quality of record keeping and inventory tracking. Functionality included within the preferred healthcare system can be split between a number of different devices, such as end-user workstations (e.g., personal computers), medication carts with computer interfaces and hand-held computerized devices (e.g., electronic tablets or personal digital assistants). The hand-held devices are preferably operated at the patient bedside. Conventional software code can be used to implement the unique functionality of the systems described herein. The code can be provided on any computer readable or usable medium.

The healthcare facility system preferably includes, or is connected to, a database that provides information relating to each of the drugs or medications that may be utilized for a patient's therapy. The system provides access to this information through a computer terminal, or other devices networked to the system. The drug file may assist the clinicians in determining which drug is appropriate for a given therapy. Additionally, the drug file may be used for purposes of tracking inventory, and for placing orders for use with a patient. The drug file may also provide information relating to administering the drug or medication.

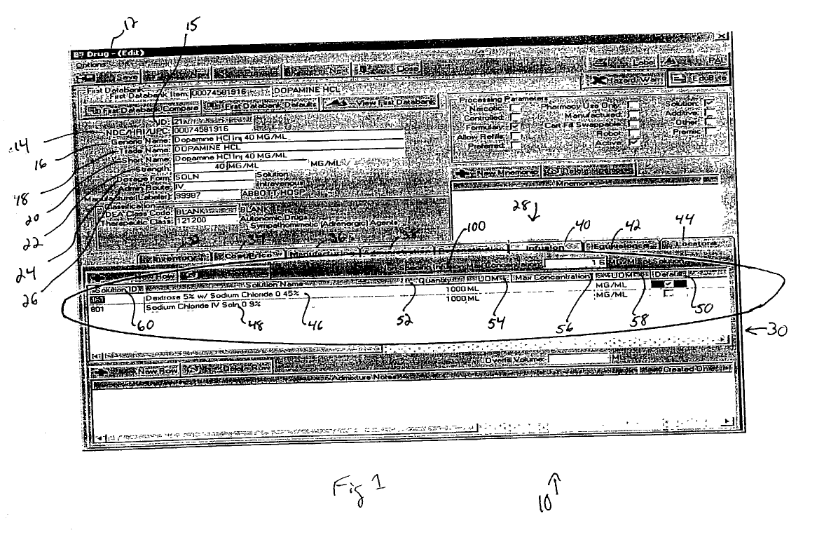

Referring to FIG. 1, a Drug (Edit) screen shot or window 12 of a drug file for a computerized healthcare facility system is shown. The Drug (Edit) window 12 can be used to provide a variety of information concerning a selected drug or medication stored in the database of the system. The window 12 may also be used for selecting a drug or otherwise ordering a drug for use in a patient's therapy (e.g., as a prescription to the pharmacy department of a hospital). Although the Drug (Edit) window 12 can provide information relating to a large variety of drugs or medications used by the healthcare facility, the present invention is concerned with those drugs or medications that are added to a diluent (i.e., a diluting agent). Such drugs or medications are referred to herein as “additives.” A typical use for an additive, is to combine the additive with a diluent for use in an infusion process for a patient. As described herein, the system is configured to identify one or more diluents that may be used for each type of additive contained in the database.

The Drug (Edit) window 12 displays a variety of information or data relating to a selected drug or additive. For example, the Drug (Edit) window 12 provides fields for an identification number 14 and hospital control numbers 15 associated with a particular drug; a generic name 16 of the drug; a trade name 18 of the drug; a short name of the drug 20; the strength 22 of the drug; the drug's dosage form 24; and an administration route 26 for the drug. In the example shown in FIG. 1, the trade name (and short name) of an additive is given as Dopamine HCL Inj 40 MG/ML. The strength is 40 MG/ML, and the dosage form is a solution. The window 12 indicates that this additive is administered via an IV (i.e., intravenous).

Additionally, in the preferred window 12 shown in FIG. 1, tabs 28 are provided to link to subwindows 30 with further information relating to the selected drug. For example, tabs 28 may be provided for inventory 32 (e.g., status and tracking of drug supplies at the facility); cost/price 34; manufacturing 36; administration 38; infusion 40; equivalence 42 and locators 44.

In accordance with the present invention, the infusion tab 40 (shown open in FIG. 1) identifies a first diluent 46 (Dextrose 5% w/Sodium Chloride 0.45%) and a second diluent 48 (Sodium Chloride IV Soln 0.9%), that can be used with the additive in a solution name box 49. Thus, a clinician desiring to administer the selected additive to a patient, can select either the first diluent or the second diluent for the IV infusion. This ability provides a degree of flexibility in the care of the patient. Although only a first and a second diluent are identified for the Dopamine additive shown in FIG. 1, other additives may identify more than two diluents that may be used.

The identified diluents in the window are preferably the recommended diluents for use with the specific additive selected. That is, such diluents are the most compatible diluents for that additive. The identified diluents are preferably approved or recommended by the Pharmacy and Therapeutic committee (i.e., P & T committee) in the healthcare facility.

The infusion tab 40 provides a Default checkbox 50 for selecting one of the identified diluents as the default diluent. Thus, a healthcare facility can designate what it considers the best or most appropriate diluent for use by making such a diluent the default.

The infusion tab 40 can also provide additional information relating to the diluent. For example, the quantity 52 and unit of measure (UOM) 54 (i.e., the default volume) can be provided.

Additionally, the maximum concentration allowed 56 may be provided. This field indicates the maximum allowed concentration of the item, which you are defining the diluent for, in a milliliter of the diluent. For example, if the user selects Dextrose 5% as one fo the diluents for Potassium Chloride, the maximum allowed concentration of the potassium in Dextrose is 80 mEq/L or 0.08 mEq/ml. This number may be different for different diluents. The unit of measure for the maximum concentration 58 can also be provided. If a max concentration for a diluent is defined, the system will use that value during order entry of the additive and diluent.

The system also provides for allowing a user (typically a designated clinician at the healthcare facility) to add a diluent to the window for an additive. First, a particular additive is selected and brought up in the Drug (Edit) window 12. A user can select a new row function to add the additional diluent. Next, the user can right click the ID box 60 or otherwise link to a list of possible diluents. The system can be configured to automatically fill the ID box 60 as well as the solution (i.e., diluent) name box 49 and the quantity box 52 upon selection of a diluent from the list.

As described, multiple solutions, default volume and specific maximum concentration can be added to the systems drug file. Additionally, a note may be associated with each diluent to help the user to decide which diluent to select and why. Such a note may include a reference to avoid the use of a particular diluent if a patient has a particular condition, such as being hypertonic.

One of the multiple diluent solutions can be marked as the default solution. The default solution will be considered the preferred solution at the time of order entry. When any additive is ordered, the system automatically validates the database and displays all related solutions. The default solution is identified but the user has the option to select any recommended alternate solutions as desired.

A number of modifications or additions can be implemented to the described system and method. For example, a nurse assisting a patient at the healthcare facility may choose the appropriate diluent in which to mix an additive at the point of care. Similarly, the pharmacist can choose an appropriate diluent at the pharmacy. In such instances the concentration checking may be done at that time. This could be the case particularly for a mix on floor admixtures.

Additionally, the system can be arranged to provide for defaulting in the diluent for the physician but allowing the pharmacy or nurse to change the default diluent prior to authorization. Healthcare facility definable policies could exist within the system for such facilities that allow this arrangement.

Although described in connection with an elaborate healthcare facility system, the functionality of the diluent identification system described herein, could also be a stand alone product. Alternatively, the diluent identification system may be part of a system that does not function like the healthcare facility system described.

While specific embodiments have been illustrated and described, numerous modifications are possible without departing from the spirit of the invention, and the scope of protection is only limited by the scope of the accompanying claims.

What is claimed is:

1. A diluent identifying system for use with a pharmacy system or healthcare facility system comprising:
a computer configured to generate information relating to a first additive for use in a patient's therapy; and,
the computer configured to generate an identification of a first diluent which may be used with the additive in the patient's therapy.

2. The diluent identifying system of claim 1 further comprising the computer configured to generate an identification of a second diluent different than the first diluent which may be used with the first additive in the patient's therapy.

3. The diluent identifying system of claim 1 further comprising the computer configured to receive information associating a second diluent which may be used with the first additive.

4. The diluent identifying system of claim 1 further comprising the computer having an interface for communication over a computer network.

5. The diluent identifying system of claim 1 further comprising the computer configured to generate a default volume for the identified first diluent.

6. The diluent identifying system of claim 1 further comprising the computer configured to generate a specific maximum concentration for use of the first additive with the first diluent.

7. The diluent identifying system of claim 2 further comprising the computer configured to generate a first note associated with the first diluent, said first note containing information relating to the first diluent.

8. The diluent identifying system of claim 7 further comprising the computer configured to generate a second note associated with the second diluent, the second note containing information relating to the second diluent.

9. The diluent identifying system of claim 2 further comprising the computer configured to receive orders for a prescription for the first additive for use in the patient's therapy.

10. The diluent identifying system of claim 9 further comprising the computer configured for receiving information from a user selecting one of said first and second diluents for completing the order for the prescription.

11. The diluent identifying system of claim 1 further comprising the computer configured for generating a plurality of additional diluents for use with the first additive in the patient's therapy.

12. The diluent identifying system of claim 1 further comprising the computer configured to generate information for a plurality of additional additives different from the first additive, and an identification of at least one diluent associated with each additional additive.

13. The diluent identifying system of claim 12 further comprising the computer configured to receive information selecting one of the first additive and additional additives for display by the computer along with the associated diluent.

14. The diluent identifying system of claim 11 further comprising a database for storing information relating to the first additive and the additional additives, and the diluents associated with the first additive and the additional additives.

15. The diluent identifying system of claim 12 further comprising the computer configured to generate an identification of additional diluents for at least one of the additional additives.

16. The diluent identifying system of claim 2 wherein the computer is configured to identify one of the first diluent and the second diluent as a default diluent.

17. A method for identifying a diluent for use with an additive in a pharmacy or healthcare facility system comprising the steps of:
providing a computer for accessing a database containing data associated with a plurality of additives for use in a patient's therapy;
receiving information by the computer selecting a first additive from said plurality of additives; and,
displaying a first diluent that can be used with the selected first additive.

18. The method of claim 17 further comprising the step of:
displaying a second diluent that can be used with the selected first additive.

19. The method of claim 17 further comprising the step of:
displaying a default volume for the first diluent.

20. The method of claim 17 further comprising the step of:
specifying a maximum concentration for the first additive when used with the first diluent.

21. The method of claim 18 further comprising the step of:
displaying a note associated with the first diluent providing information relating to the first diluent.

22. The method of claim 21 further comprising the step of:
displaying a note associated with the second diluent providing information relating to the second diluent.

23. The method of claim 18 further comprising the step of:
identifying one of the first diluent and the second diluent as the default diluent.

24. The method of claim 17 further comprising the steps of:
inputting a second diluent associated with the first additive into the database.

25. A computer program product for identifying a diluent for use with an additive, said computer program product comprising:
a computer usable medium having computer readable program code embodied therein, the program code including
a first portion for maintaining data relating to a plurality of additives for use in a patient's therapy; and,
a second portion for identifying a first diluent for use with each of the plurality of additives.

26. The computer program product of claim 25 further comprising a third portion for identifying a second diluent for at least one of the plurality of additives.

27. The computer program product of claim 25 further comprising a third portion for identifying a second diluent for each of the plurality of additives.

28. The computer program of claim 25 further comprising a third portion for indicating a default volume for the first diluent.

29. The computer program of claim 25 further comprising a third portion for indicating a specific maximum concentration for use of the first diluent with one the plurality of additives.

30. The computer program of claim 25 further comprising a third portion for generating a note associated with the first diluent, the note providing data relating to the first diluent.

31. The computer program of claim 26 further comprising a fourth portion for generating a note associated with the second diluent, the note providing data relating to the second diluent.

32. The computer program of claim 26 further comprising a fourth portion for setting one of the first diluent and the second diluent as a default solution for use with at least one of the additives.
